(19) United States
(12) Patent Application Publication
(10) Pub. No.: US 2004/0161445 A1
(43) Pub. Date: Aug. 19, 2004

(54) Hearing aid or hearing aid components for placement in the auditory canal and/or the auricle of a wearer

(75) Inventors: Michael Bulk, Herford (DE); Michael Rohr, Enger (DE); Helmut Schmidt, Saarbrucken-Gudingen (DE); Carsten Becker-Willinger, Saarbrucken (DE)

Correspondence Address: BOURQUE & ASSOCIATES, P.A., 835 HANOVER STREET, SUITE 303, MANCHESTER, NH, 03104, US

(73) Assignee: AS Audio Service GmbH

(21) Appl. No.: 10/783,537

(22) Filed: Feb. 20, 2004

(30) Foreign Application Priority Data
Mar. 27, 2003 (WO) PCT/EP03/03165
May 2, 2002 (DE) 102 19 679.6

(51) Int. Cl. A61K 33/38; H04R 25/00

(52) U.S. Cl. 424/423; 381/322; 424/618

(57) ABSTRACT

A hearing aid or hearing aid component for placement in an auditory canal and/or in or behind an auricle of a wearer is provided with a biofilm-inhibiting coating. This biofilm-inhibiting coating includes an inorganic condensate that is modified with organic groups on the basis of a coating composition, which includes a hydrolysate or pre-condensate of one or more hydrolysable compounds with at least one non-hydrolysable substituent, whereby at least one portion of the organic groups of the condensate includes fluorine atoms and/or copper or silver colloids contained in the coating.

### TECHNICAL FIELD OF THE INVENTION

The invention relates to a hearing aid or hearing aid component and more particularly, to a hearing aid or hearing aid component intended for placement in an auditory canal and/or in or behind an auricle of a wearer.

### DESCRIPTION OF THE RELATED ART

Hearing aids or hearing aid components that are adapted to the auricle and/or the auditory canal are, at times, also called otoplastics. However, in the industry, it has become common to use the term otoplastic only for that part of a behind-the-ear device (BTE device) that is placed in the auditory canal. According to the habitual language use of recent times, the term “otoplastic” is too narrow for the subject of the invention. Rather, the subject of the invention deals with all hearing aids and hearing aid components that come into contact with the skin of the wearer.

BTE devices are typically electrical or electronic devices and include a microphone, an amplifier, a loudspeaker and possibly other electronics, such as a microprocessor, in order to improve hearing. In addition to BTE devices, where the electrical or electronic component is worn behind the ear and connected to an ear plug located in the auditory canal and today is designated as otoplastic, via a tube, the most widely used hearing aids are so-called in-the-ear hearing aids. With such in-the-ear hearing aids that are to be built as small as possible, the entire hearing aid is enclosed in a shell and its outer contours correspond to the inner contours of the auditory canal.

Providing hearing aids of the kind described above is often problematic. Wearing the hearing aids can trigger various undesired reactions. In addition to inflammation or allergic reactions, which may be counteracted through continuous, very careful cleaning of the contact areas of the outer surface of the hearing aids, there are problems with patients who suffer from otitis externa. Currently, patients with this disease cannot be provided with hearing aids at all.

Cerumen, commonly known as ear wax, forms in the auditory canal. This cerumen as well as other excretions may be deposited on the contact areas of the hearing aid or hearing aid components. This leads to a coating, which is also called biofilm, where microorganisms may nest. Because of this occurrence, the contact areas of hearing aids or hearing aid components with the skin of the wearer must be cleaned often to remove the biofilm. Because the biofilm is difficult to remove and re-builds relatively quickly, after extensive use, the buildup of biofilm cannot be avoided with sufficient surety.

### SUMMARY OF THE INVENTION

Based on the state-of-the-art explained above, it is an aspect of the invention to create a hearing aid or hearing aid components wherein said hearing aid or hearing aid components are very skin-friendly, even for problem patients, and wherein the areas that are in contact with the skin of the wearer can be cleaned and kept clean easily. In particular, the formation of biofilm shall be avoided.

The solution to this aspect is carried out by a hearing aid or a hearing aid component for placement in the auditory canal and/or in or behind the auricle of a wearer, with a biofilm-inhibiting coating of an inorganic condensate modified with organic groups on the basis of a coating composition, which includes a hydrolysate or pre-condensate of one or more hydrolysable compounds with at least one non-hydrolysable substituent, whereby at least one part of the organic groups of the condensate exhibits fluorine atoms and/or copper or silver colloids are contained in the coating.

### DETAILED DESCRIPTION

The present invention includes a hearing aid or hearing aid component for placement in the auditory canal and/or in or behind the auricle of a wearer, with a biofilm-inhibiting coating of an inorganic condensate modified with organic groups on the basis of a coating composition, which includes a hydrolysate or pre-condensate of one or more hydrolysable compounds with at least one non-hydrolysable substituent, whereby at least one part of the organic groups of the condensate exhibits fluorine atoms and/or copper or silver colloids are contained in the coating. Surprisingly, the application of a coating of inorganic condensate of the kind just described and modified with organic groups provides dramatic improvements in solving the problems mentioned above in the Description of the Related Art. Coating the surface of hearing aids or hearing aid components with such a material has the result that the formation of biofilm on the hearing aid or the hearing aid component during use is significantly inhibited or may even be avoided entirely. The respective coating also makes it possible to provide hearing aids to problem patients, such as patients suffering from otitis externa, who until now could not be provided with hearing aids.

By applying a coating to the external surface of the hearing aid or hearing aid component, the risk of inflammation in-the-ear or at the auricle is reduced significantly, as is the risk of allergic reactions. The use of condensates with organic groups that exhibit, at least partially, fluorine atoms, avoids in particular the accretion of the biofilm on the otoplastic due to the anti-adhesion and/or easy-to-clean properties. The presence of copper or silver colloids in the coating prevents in particular the continued growth of a biofilm due to the microbicide effect. Through a combination of condensates with fluorine-containing organic groups and the presence of copper or silver colloids in the coating both the biofilm inhibiting effect and the anti-allergic effect are improved in a synergetic manner, such that such a combination presents a particularly preferred embodiment.

The hearing aids or hearing aid components, which are provided with the external coating, can be made of any suitable material, even those already in use. Typically, these are synthetic materials, preferably polymethyl methacrylate (PMMA). Examples of other suitable synthetics are polyethylene, polypropylene, polyacrylates such as polymethyl acrylate, polyvinyl butyral or polycarbonate. The casings of the hearing aids or hearing aid components may be of a single material or assembled with several parts of different materials. Before the application of the coating, the surfaces of the hearing aid casings or hearing aid components can be partially or completely glazed, gold-plated or electroplated. The coatings are used especially for the components or the areas of the hearing aid that are placed in the auditory canal.

The coating may be applied to the entire surface of the hearing aid or hearing aid component. The coating does not need to be applied to non-critical areas, such as where biofilm formation or contact with the skin of the wearer is not expected. Even though it is possible to coat the inside areas of a hollow shell, a coating does not need to be applied. It may also be sufficient to coat only parts of the hearing aids or the hearing aid components.

The hearing aids or hearing aid components can be pre-treated in a typical manner, e.g., cleaned or degreased, to achieve a good bond with the coating to be applied. Of course, if only a partial area of the hearing aid or of the hearing aid component is to be coated, the partial area or the respective part is initially coated separately and then added to the finished device or device component. In the preferred embodiment, the area to be coated on the hearing aid or the hearing aid component is initially treated with an adhesion promoter or a primer. The coating may be obtained by applying a coating composition that will be explained below to the hearing aid or hearing aid component and subsequent treatment with heat and/or radiation. In the preferred embodiment, light-curing coating compositions are used.

The coating composition (lacquer) used includes a hydrolysate or pre-condensate on the basis of one or several hydrolysable compounds with at least one non-hydrolysable substituent. The non-hydrolysable substituent is, in particular, an organic substituent (carbonaceous). Preferably, the coating composition includes at least one hydrolysable silane with at least one non-hydrolysable substituent as hydrolysable compound.

The hydrolysate or pre-condensate is preferably obtained through hydrolysis or condensation from one or more silanes of the general formula (I)

RₐSiX₍₄₋ₐ₎ (I)

wherein the residues R are analogous or different and represent non-hydrolysable groups, the residues X are analogous or different and represent hydrolysable groups or hydroxyl groups and a has a value of 1, 2 or 3, but preferably the value 1.

With the organosilanes of formula (I), the hydrolysable groups X are, for example, hydrogen or halogen (F, Cl, Br or I, in particular Cl and Br), alcoxy (preferably C₁₋₆-alcoxy, in particular Cl₁₋₄-alcoxy, such as, for example, methoxy, ethoxy, n-propoxy, i-propoxy, n-butoxy, i-butoxy, sec.-butoxy and tert.-butoxy), aryloxy (preferably C₆₋₁₀-aryloxy, such as, for example, phenoxy), acyloxy, (preferably C₁₋₆-acyloxy, such as, for example, acetoxy or propionyloxy), alkylcarbonyl (preferably C₂₋₇-alkylcarbonyl such as, for example, acetyl), amino, monoalkylamino or dialkylamino, where the alkyl groups exhibit preferably 1 to 12, in particular 1 to 6 carbon atoms. Preferred hydrolysable residues are halogen, alcoxy groups and acyloxy groups. Particularly preferred hydrolysable residues are alcoxy groups, in particular methoxy and ethoxy.

R is a non-hydrolysable organic residue, which may carry a functional group. Examples for R are alkyl (preferably C₁₋₆-alkyl, such as methyl, ethyl, n-propyl, isopropyl, n-butyl, s-butyl and t-butyl, pentyl, hexyl or cyclohexyl), alkenyl (preferably C₂₋₆-alkenyl, such as e.g., vinyl, 1-propenyl, 2-propenyl and butenyl), alkinyl (preferably C₂₋₆-alkinyl, such as, for example, acetylenyl and propargyl) and aryl (preferably C₆₋₁₀-aryl, such as, for example, phenyl and naphtyl).

Exemplary functional groups of the residue R are, in addition to the groups already mentioned above with unsaturated C-C-bonds, the epoxy, hydroxy, ether, amino, monoalkylamino, dialkylamino, for example, with the above defined C₁₋₆-alkyl groups, amid, carboxy, mercapto, thioether, vinyl, isocyanate, acryloxy, methacryloxy, acid anhydride, acid halogenide, cyano, halogen, aldehyde, alkylcarbonyl, sulphonic acid and phosphoric acid group. These functional groups are bonded to the silicon atom via alkylene, alkenylene or arylene bridge groups, which may also be interrupted by oxygen or sulphur atoms or NH groups. The mentioned bridge groups are derivates, for example, of the alkyl, alkenyl or aryl residues mentioned above. The residues R contain preferably 1 to 18, in particular 1 to 8 carbon atoms. The mentioned residues R and X may possibly include one or more typical substituents, such as, for example, halogen or alcoxy.

Preferably, at least one of the hydrolysable silanes with at least one non-hydrolysable substituent contains at least one of the aforementioned functional groups at the non-hydrolysable substituents. Cross-linking can then occur via these functional groups, e.g., through a reaction of the functional groups among each other at the silanes, whereby different or analogous functional groups may react with one another, or with functional groups at the organic compounds described below that may also be contained in the coating composition. Cross-linking via the functional groups leads to curing via the organic groups contained in the condensate (organic cross-linking).

Preferred functional groups are carbon-carbon double bonds as well epoxy, acid anhydride and amino groups, whereby the use of carbon-carbon double bonds as functional groups is particularly preferred.

The employed compounds with carbon-carbon double bonds as functional groups are in particular silanes of the general formula (I), wherein the residue R includes a reactive polymerizable double bond. Preferred is a silane of the general formula (I), wherein X and a are as defined above (X is preferably methoxy or ethoxy, a is preferably 1) and R is a non-hydrolysable residue, e.g., an aliphatic, cycloaliphatic or aromatic residue, in particular alkylene, e.g., C₁-C₆-alkylene, such as methylene, ethylene, propylene and butylene with a vinyl, (meth)acryl or (meth)acryloxy group. The residue R is preferably a (meth)acryloxy-(C₁₋₆)-alkylene residue, such as, for example, (meth)acryloxy-propyl. Examples are vinyltriethoxysilane, vinyltrimethoxysilane, methacryloxypropyltrimethoxysilane (MPTS), methacryloxypropyltriethoxysilane, acryloxypropyltrimethoxysilane, and acryloxypropyltriethoxysilane.

Examples of silanes with an epoxide group are epoxy silanes of the aforementioned general formula (I), wherein a has a value of 1, X is preferably C₁₋₄-alcoxy, and in particular preferred are methoxy and ethoxy, and R is a non-hydrolysable residue with at least one epoxide group, e.g., an aliphatic, cycloaliphatic or aromatic residue, in particular alkylene, e.g., C₁-C₆-alkylene, such as methylene, ethylene, propylene and butylene with at least one epoxide group. The residue R is preferably a glycidyloxy-(C₁₋₆)-alkylene residue, such as, for example, γ-glycidyloxypropyl. Examples are γ-glycidyloxypropyltrimethoxysilan (GPTD) and γ-glycidyloxypropyltriethoxysilan (GPTES).

Examples of aminosilanes are those of the aforementioned general formula (I), wherein a has a value of 1, X is preferably C₁₋₄-alcoxy, and preferably methoxy and ethoxy, and R is a non-hydrolysable residue with at least one amino group, e.g., an aliphatic, cycloaliphatic or aromatic residue, in particular alkylene, e.g., C₁-C₆-alkylene, such as methylene, ethylene, propylene and butylene with at least one primary, secondary or tertiary amino group. For example, R is a R¹₂N-(alkylene-NR¹)ₓ-alkylene residue, where x is 0 to 5, where the alkylene groups can be analogous or different and can be in particular the aforementioned ones, and wherein R¹ is analogous or different and is hydrogen or possibly a substituted alkyl residue, e.g., the ones mentioned in the above general formula (I). R¹ may also be a bivalent residue, e.g., alkylene, under formation of a heterocyclic ring. Furthermore, an additional, non-hydrolysable residue, e.g., alkyl may be present as well (a=2). Examples of such silanes are 3-aminopropyltrimethoxysilane (APTS), 3-aminopropyltriethoxysilane, N-(2-aminoethyl)-3-aminopropyltrimethoxysilane, N-[N′-(2′-aminoethyl)-2-aminoethyl]-3-aminopropyltrimethoxysilane, N-[3-(triethoxysilyl)-propyl]-4,5-dihydroimidazol and [N-(2-aminoethyl)-3-aminopropyl]-methyldiethoxysilane.

Examples of anhydridesilanes are those of the aforementioned general formula (I), wherein a has a value of 1, X is preferably C₁₋₄-alcoxy, and preferably methoxy and ethoxy, and R is a non-hydrolysable residue with at least one anhydride group, e.g., an aliphatic, cycloaliphatic or aromatic residue, in particular alkylene, e.g., C₁-C₆-alkylene, in particular C₁-C₄-alkylene such as methylene, ethylene, propylene and butylene with one anhydride group. The anhydride group, that just like the epoxide group is capable for condensation with amino groups, may be, for example, residues that derive from carbon acid anhydrides such as succinic acid anhydride, maleic acid anhydride or phthalic acid anhydride, which are connected to the silicon atom via one of the aforementioned residues, in particular C₁-C₄-alkylene. Examples are [3-(triethoxysilyl)propyl] succinic acid anhydride, (dihydro-3-(3-triethoxysilyl)propyl)-2,5-furandione, GF20) and [3-(trimethoxysilyl)propyl] succinic acid anhydride.

According to one embodiment of the invention, the biofilm-inhibiting coating is at least a portion of the organic groups of the inorganic condensate with fluorine substituted.

For this purpose, the hydrolysable compounds employed in the coating composition comprise one or more hydrolysable silanes with at least one fluorine-containing non-hydrolysable group. Such silanes are described in detail, for example, in WO 92/21729 or in DE 4118184, which are herewith referenced and which are hereby incorporated by reference. Use of such a fluorinated silane leads to hydrophobic and oleophobic properties of the coating.

Preferably, hydrolysable silane compounds with at least one non-hydrolysable residue can be employed for this purpose, which exhibit the general formula

Rf(R)_{b}SiX_{(3-b)} (II)

wherein X and R are defined as in formula (I), Rf is a non-hydrolysable group, that exhibits 1 to 30 fluorine atoms bound to carbon atoms, which are preferably separated from Si by at least two atoms, preferably an ethylene group, and b is 0, 1 or 2. R is, in particular, a residue without a functional group, preferably an alkyl group such as methyl or ethyl. Preferably, the Rf groups contain at least 2, 3, 5 or 8 fluorine atoms but not more than 25, 21 or 18 fluorine atoms that are bound to aliphatic or cycloaliphatic carbon atoms. Rf is preferably a fluorinated alkyl group with 3 to 20 C-atoms and examples are CF₃CH₂CH₂, C₂F₅CH₂CH₂, n-C₆F₁₃CH₂CH₂, i-C₃F₇OCH₂CH₂CH₂, n-C₈F₁₇CH₂CH₂ and n-C₁₀F₂₁—CH₂CH₂.

Fluorine atoms that may be bound to aromatic carbon atoms (e.g., in the case of C₆F₄) are not taken into account. The fluorine-containing group Rf may also be a chelating ligand. It is also possible that one or more fluorine atoms are present at a carbon atom, from which a double or triple bond originates. Examples of usable fluorine silanes are CF₃CH₂CH₂SiCl₂(CH₃), CF₃CH₂CH₂SiCl (CH₃)₂, CF₃CH₂CH₂Si(CH₃) (OCH₃)₂, C₂F₅CH₂CH₂—SiZ₃, n-C₆F₁₃—CH₂CH₂SiZ₃, n-C₈F₁₇—CH₂CH₂—SiZ₃, n-C₁₀F₂₁—CH₂CH₂—SiZ₃, where Z=OCH₃, OC₂H₅ or Cl; i-C₃F₇O—CH₂CH₂CH₂—SiCl₂(CH₃), n-C₆F₁₃—CH₂CH₂—Si(OCH₂CH₃)₂, n-C₆F₁₃—CH₂CH₂—SiCl₂(CH₃), and n-C₆F₁₃—CH₂CH₂—SiCl(CH₃)₂.

If fluorine-containing organic groups are contained in the condensate, typically not more than 0.1 mole percent, in particular not less than 0.5 mole percent, preferably not less than 1 mole percent, more preferred not less than 2 mole percent, and most preferred not less than 4 mole percent and typically 100 mole percent or less, in particular not more than 50 mole percent, preferably not more than 30 mole percent and more preferred not more than 15 mole percent of all non-hydrolysable groups of the hydrolysable compounds employed in the coating composition are groups that include one or more fluorine atoms. These portions are also preferred when additional copper or silver colloids are contained in the coating.

Of the employed hydrolysable silanes with at least one non-hydrolysable substituent for the hydrolysate or pre-condensate, preferably at least 40 mole percent, preferably at least 70 mole percent, particularly preferred at least 90 mole percent exhibit at least one functional group at at least one non-hydrolysable substituent. In one preferred embodiment, with the exception of possibly employed fluorine-containing silanes, all other employed hydrolysable silanes with at least one non-hydrolysable substituent at at least one non-hydrolysable substituent possess at least one functional group that allow cross-linking.

For the production of the hydrolysate or the pre-condensate, additional hydrolysable compounds of an element M without non-hydrolysable groups can be employed as matrix formers. They are, in particular, compounds of glass-forming or ceramic-forming elements, in particular compounds of at least one element M from the main groups III to V and/or the secondary groups II to IV of the periodic table or periodic system of elements. Preferably, they are hydrolysable compounds of Si, Al, B, Sn, Ti, Zr, V or Zn, in particular those of Si, Al, Ti or Zr, or mixtures of two or more of these elements. It shall be noted in this regard that other hydrolysable compounds may, of course, be employed as well, in particular those of elements of the main groups I and II of the periodic table or periodic system (e.g., Na, K, Ca and Mg) and of the secondary groups V to VIII of the periodic table or periodic system (e.g., Mn, Cr, Fe and Ni). Hydrolysable compounds of the lanthanides can be used as well. Preferably, these hydrolysable compounds without non-hydrolysable groups, however, do not constitute more than 40 and in particular not more than 20 mole percent and in particular 0 mole percent of the entire employed hydrolysable monomeric compounds. When employing highly reactive hydrolysable compounds (e.g., aluminum compounds) it is recommended to use complexing agents, which prevent spontaneous precipitation of the respective hydrolysates after adding water. WO 92/21729, which is hereby incorporated by reference, mentions suitable complexing agents that can be employed with reactive hydrolysable compounds.

These compounds exhibit, in particular, the general formula MXn, where M is the previously defined element, X is defined as in formula (I), whereby two groups X can be replaced by one oxo group, and n corresponds to the valence of the element and is typically 3 or 4. Preferably, alkoxides of Si, Zr or Ti are used. Coating compositions on the basis of hydrolysable compounds with non-hydrolysable groups and hydrolysable compounds without non-hydrolysable groups are described, e.g., in WO 95/31413 (DE 4417405), which are herewith referenced and which are hereby incorporated by reference.

Additional suitable compounds without non-hydrolysable groups are, in particular, hydrolysable silanes, that exhibit, for example, the formula

SiX₄ (III)

wherein X is defined as in formula (I). Examples are Si (OCH₃)₄, Si (OC₂H₅)₄, Si(O-n or i-C₃H₇)₄, Si(OC₄H₉)₄, SiCl₄, HSiCl₃, Si(OOCC₃H)₄. Of these silanes, tetramethoxysilane and tetraethoxysilane are particularly preferred.

In additional embodiments of the invention, copper or preferably silver colloids are contained in the biofilm-inhibiting coating. To this end, respective copper or silver colloids can be introduced into the coating composition, such that after applying the coating composition and after drying, or curing, respectively, a coating with copper or silver colloids is formed in the inorganic condensate matrix, which is modified with organic groups.

However, the copper or silver colloids are preferably formed in the coating composition in situ from copper or silver compounds. This can be done through heat and/or radiation treatment, whereby the treatment can be carried out prior to the application or preferably after the application of the coating composition, that is, together with drying or curing of the coating, or prior to and after the application.

In this case, the coating composition comprises at least one copper or silver compound. These can be copper or silver compounds that are soluble in water or in organic solvents, e.g., AgNO₃ or CuO₄; however, the copper or silver ions are preferably introduced in the form of complex compounds and in particular of chelate complex compounds. Under reducing conditions, the copper or silver (I) ions or the copper or silver complex compounds can react to metal colloids. Examples of complexing agents that form a copper or silver complex using copper or silver (I) ions are halogenide ions such as iodide, bromide and, in particular, chloride (or the respective halogen hydracids), thio compounds, thiocyano compounds, sugars such as pentose and hexose, e.g., glucose, β-dacarbonyl compounds such as diketone, e.g., acetylacetonate, keto ester, e.g., acetic acid ester and allylacetoacetate, ether alcohols, carboxylic acid, carboxylates, e.g., acetate, citrate or glycolate, betaines, dioles, polyoles, also polymers such as polyalkylene glycols, crown ether, phosphorus compounds and amino compounds. Especially preferred is the use of amino compounds such as amino silanes, mono-, di-, tri-, tetraamines and higher polyamines as complexing agents. Examples of organic amines are triethylenetetramine, diethylenetriamine and ethylenediamine. Examples of aminosilanes are 3-aminopropyltri(m)ethoxysilane and in particular 2-aminoethyl-3-aminopropyltrimethoxysilane (DIAMO), 2-aminoethyl-3-aminopropyltriethoxysilane, aminohexyl-3-aminopropyltrimethoxysilane and aminohexyl-3-aminopropyltriethoxysilane. Preferably employed are copper diamine or silver diamine complex compounds, whereby complexing agents with at least two amino groups, which can form chelate complexes, are particularly well suited. Particularly preferred of the amino complexing agents are the amino silanes. Preferably, they are integrated into the forming matrix, which can contribute to the stabilization of the copper or silver colloids.

When using a complexing agent, the stoichiometric ratio of either Cu or Ag to the present complexing groupings is preferably 1:0.1 to 1:500, in particular, 1:1 to 1:200. The complexing agents may also function, at least partially, as reducing agents for the copper or silver ions. In addition, the solvents described below, e.g., alcohols or ketones, the by-products that form during hydrolysis and condensation, e.g., alcohols, the employed hydrolysable compounds or a combination of those may be considered as reducing agents.

In a preferred embodiment, the coating composition may also contain nanoscale inorganic solid particles, resulting in an increased mechanical strength (scratch resistance, hardness) of the coating. Since with lengthy use, potential scratches may aid in the formation of biofilm due to unevenness, these nano-particles support the biofilm-inhibiting behavior as well.

In general, they have a particle size in a range of 1 to 300 nm or 1 to 100 nm, preferably 2 to 50 nm and in particular preferred between 5 and 20 nm. This material may be introduced in the form of a powder, however its preferred use is in the form of an acid or alkaline stabilized sol. The nanoscale inorganic solid particles may be made up of any inorganic materials, however, they consist in particular of metals or metal compounds such as, for example, (possibly hydrated) oxides, such as ZnO, CdO, SiO₂, TiO₂, ZrO₂, CeO₂, SnO₂, Al₂O₃, In₂O₃, La₂O₃, Fe₂O₃, Cu₂O, Ta₂O₅, Nb₂O₅, V₂O₅, MoO₃ or WO₃, chalcogenides, nitrides, phosphides, phosphates, silicates, zirconates, aluminates or carbides. The nanoscale inorganic solid particles are preferably oxides, oxide hydrates, nitrides or carbides of Si, Al, B, Zn, Cd, Ti, Zr, Ce, Sn, In, La, Fe, Cu, Ta, Nb, V, Mo or W, in particular preferred are those of Si, Al, B, Ti and Zr. Particularly preferred is the use of oxides or oxide hydrates. Preferred nanoscale inorganic solid particles are SiO₂, Al₂O₃, ITO, ATO, AlOOH, ZrO₂, and TiO₂. Examples of nanoscale SiO₂ particles are commercially available silicic acid products, e.g., silica sol, such as Levasil®, silica sol of Bayer AG, or pyrogenic silicic acids, e.g., the Aerosil products of Degussa.

The nanoscale inorganic solid particles may be nanoscale inorganic solid particles modified with organic surface groups. The surface modification of nanoscale solid particles is a known method as described, for example, in WO 93/21127 (DE 4212633) and WO 98/51747 (DE 19746885), which are hereby incorporated by reference.

The nanoscale inorganic solid particles may be employed in an amount of 1 to up to 50 percent in weight, relative to the solids components of the coating composition. In general, the content of nanoscale inorganic solid particles is in a range of 1 to 30 percent in weight.

The coating composition may contain other additives, which are typically added depending on purpose and desired properties. Examples are organic compounds, cross-linking agents, solvents, organic and inorganic color pigments, coloring agents, UV absorbers, slip agents, leveling agents, wetting agents, adhesion promoter and initiators. Initiators may be used for thermally or photochemically induced cross-linking.

Organic compounds or cross-linking agents may be added to the coating composition. These may be organic monomers, oligomers or polymers, which, in particular, contain at least two functional groups that may react with the functional groups of the employed hydrolysable silanes under formation of an organic cross-linking. These may be, for example, aliphatic, cycloaliphatic or aromatic compounds. Preferred is the use of organic compounds with at least two carbon-carbon double or triple bonds, at least two epoxy groups or at least two amino groups, whereby the use of carbon-carbon double bonds is particularly preferred. Examples of such organic compounds are compounds with at least two acryloxy, methacryloxy glycidyloxy, epoxy, hydroxyl and/or amino groups. The organic compounds are preferably used in an amount of not more than 30 percent in weight, relative to the solids content of the coating composition.

Examples of organic compounds with at least two carbon-carbon double bonds are 1,6-hexanediol-dimethacrylate, 1,6-hexanediol diacrylate, bisphenol A-bisacrylate, bisphenol A-bismethacrylate, trimethylolpropane triacrylate, trimethylolpropane trimethacrylate, neopentyl glycol dimethacrylate, neopentyl glycol diacrylate, diethylene glycol diacrylate, diethylene glycol dimethacrylate, triethylene glycol diacrylate, diethylene glycol dimethacrylate, tetraethylene glycol diacrylate, tetraethylene glycol dimethacrylate, polyethylene glycol diacrylate, polyethylene glycol dimethacrylate, 2,2,3,3-tetrafluor-1,4-butanediol diacrylate and dimethacrylate, 1,1,5,5-tetrahydroper fluorpentyl-1,5-diacrylate and -dimethacrylate, hexafluorine bisphenol A-diacrylate and -dimethacrylate, octafluorine hexandiol-1,6-diacrylate and -dimethacrylate, 1,3-to (3-methacryloxypropyl)tetrakis(trimethylsiloxy)-disiloxane, 1,3-to (3-acryloxypropyl)tetrakis(trimethylsiloxy)disiloxan, 1,3-to(3-methacryloxypropyl)tetramethyldisiloxane and 1,3-to(3-acryloxypropyl)tetramethyldisiloxane.

Usable organic epoxy compounds may be derived, for example, from aliphatic, cycloaliphatic or aromatic esters or ethers or mixtures thereof, e.g., on the basis of ethylene glycol, 1,4-butanediol, propylene glycol, 1,6-hexanediol, cyclohexane dimethanol, pentaerythrite, bisphenol A, bisphenol F or glycerin. Examples of organic compounds with at least two epoxy groups are 3,4-epoxycyclohexylmethyl-3,4-epoxycyclohexanecarboxylate, bis-(3,4-epoxycyclohexyl)adipate, 1,4-butanediol glycide ether, cyclohexane dimethanol diglycide ether, glycerin triglycide ether, neopentyl glycol diglycide ether, pentaerythrite polyglycide ether, 2-ethyl hexylglycide ether, 1,6-hexane dialdiglycide ether, propylenegycol diglycide ether, polypropyleneglycol diglycide ether, bisphenol-A-diglycide ether, bisphenol-F-diglycide ether, epoxy resins on the basis of bisphenol A, epoxy resins on the basis of bisphenol F and epoxy resins on the basis of bisphenol A/F. Examples of organic compounds with at least two amino groups are 1,3-diaminopentane, 1,5-diamino-2-methylpentane, 1,4-diaminocyclohexane, 1,6-diaminohexane, diethylenediamine, triethylenetetramine or isophorondiamine. Organic compounds that carry various functional groups may, of course, be used as well.

All initiators/initiating systems known to the specialists or those skilled in the art, including radical photo initiators, radical thermal initiators, cationic photo initiators, cationic thermal initiators, and any combination thereof may be considered as initiators or cross-linking agents.

Examples of employable radical photo initiators are Irgacure® 184 (1-hydroxycyclohexyl phenyl ketone), Irgacure® 500 (1-hydroxycyclohexyl phenyl ketone, benzophenone) and other photo initiators available from the Ciba-Geigy company of the Irgacure® type; Darocure® 1173, 1116, 1398, 1174, and 1020 (available from the Merck company), benzophenone, 2-chlorine-thioxanthone, 2-methyl-thioxanthone, 2-isopropyl-thioxanthone, benzoin, 4,4′-dimethoxybenzoin, benzoin ethylene ether, benzoin isopropyl ether, benzil dimethyl ketal, 1,1,1-trichloride acetophenon, diethoxy-acetophenone and dibenzosuberone.

Examples of radical thermal initiators are, among others, organic peroxides in the form of diacyl peroxides, peroxide carbonates, alkyl peresters, alkyl peroxides, perketales, ketone peroxides, and alkyl hydroperoxides, as well as azo compounds. Examples of these are, in particular, dibenzoyl peroxide, tert-butyl perbenzoate and azobisisobutyronitrile.

An example of a cationic photo initiator is Cyracure® UVI-6974, while 1-methylimidazole is a preferred cationic thermal initiator.

These initiators are used in the typical amounts known to specialists or those skilled in the art, preferably 0.01 to 5 percent in weight, and preferably 0.1 to 3 percent in weight relative to the overall solids content of the coating composition.

Examples of suitable solvents are alcohols, preferably low aliphatic alcohols such as methanol, ethanol, 1-propanol, i-propanol, and 1-butanol, ketones, preferably low dialkyl ketones such as acetone and methylisobutyketone, ethers, preferably low dialkyl ethers such as diethyl ether, dibutyl ether and THF, isopropoxyethanol, aromatic hydrocarbons such as toluol, esters such as ethyl acetate, butoxyethanol, sulfoxides, sulfones, amides such as dimethylformamide and their mixtures. Essentially, there is no need to use a solvent, especially if the hydrolysis of the hydrolysable silanes leads to the formation of alcohols such as the ones mentioned above, for example. Of course, a solvent may be used even in such cases.

The hydrolysis or the (pre-)condensation of the hydrolysable compounds occurs in particular according to the sol-gel method. The sol-gel method is known to the specialist or those skilled in the art. The hydrolysis or condensation is performed either in the absence of a solvent or preferred in an aqueous or aqueous/organic reaction medium, possibly in the presence of an acid or alkaline condensation catalyst such as HCl, HNO₃ or NH₃. The result is a partial hydrolysis or (poly-)condensation of the hydrolysable compounds (hydrolysate or pre-condensate). The degree of condensation can be set in the same manner as the viscosity in a suitable manner, e.g., through solvents. Additional details concerning the sol-gel method are described, for example, at C. J. Brinker, G. W. Scherer: “Sol-Gel Science—The Physics and Chemistry of Sol-Gel-Processing,” Academic Press, Boston, San Diego, New York, Sydney (1990), which is hereby incorporated by reference. The sol obtained in this manner is employed as a coating composition, to which may be added in any order additional components such as copper or silver complex compounds or the nanoscale particles. Such components may also be mixed with the hydrolysable compounds prior to or during the hydrolysis or pre-condensation. It is, furthermore, possible that initially a hydrolysable compound is hydrolyzed or pre-condensated, and additional employable hydrolysable compounds, such as fluorine silanes, are added later.

The coating composition can be applied to the otoplastic using any typical manner. All common wet chemical coating methods can be employed. Examples are centrifugal coating, (electric) dip coating, blade coating, spray coating, spin coating, drawing, centrifuging, casting, rolling, painting, flood coating, foil casting, knife coating, slot coating, meniscus coating, curtain coating, roller application or typical printing methods such as screen printing or flex printing. The amount of the applied coating composition is selected such that the desired coating thickness is achieved. For example, it is done such that dry coating thickness in a range of 1 to 15 μm and preferably in a range of 2 to 5 μm are achieved. An advantage of the present invention is the variably adjustable coating thickness. After the coating composition is applied, drying, for example at ambient temperatures (under 40° C.), may be carried out.

The potentially pre-dried coating is generally subjected to a treatment with heat and/or radiation to cure the coating. In one preferred embodiment, curing is carried out by radiation. Actinic radiation, e.g., UV or laser radiation, or electron radiation is used for the radiation. Particularly preferred is radiation using UV radiation or blue light, such as is used in the dental field. Through the radiation, copper or silver compounds that are utilized can be converted to copper or silver colloids.

Surprisingly, colloids are formed in the coating composition from the copper or silver compounds already at relatively low temperatures. The formation of colloids starts in particular at temperatures of under 200° C., in particular under 130° C., under 100° C. and even already under 80° C.; in general, a temperature of more than 50° C. is required if no radiation is carried out. The colloid formation starts, for example, at a heat treatment in a range of 50 to 100° C., preferably of 60 to 80° C. or 70 to 80° C. In a preferred embodiment, such a heat treatment of the coating composition can occur even prior to the application of the coating composition in order to initiate or to complete the colloid formation. During the subsequent treatment of the applied coating with heat or preferably with radiation, the colloid formation can be continued if it has not been completed already.

Larger colloids that have a long-term effect and have diameters of, for example, 5-20 nm, in particular of 10-20 nm, can be formed. Surprisingly, it has been shown that through radiation and/or heat treatment, copper or silver colloids with diameters of, for example, 10 to 30 nm can be formed rather rapidly. The amount of copper or silver colloids introduced into the coating composition depends on the desired concentration of colloids in the coating and can be several percent in weight, for example.

Curing of the coating composition can also be carried out through heat treatment at temperatures of less than 300° C., preferably not more than 200° C., and in particular not more than 130° C. Preferred are temperatures that are also suited for the aforementioned colloid formation, e.g., below 100° C. or below 80° C., e.g., 50 to 100° C. or 60 to 80° C. Of course, this heat treatment will also lead to a colloid formation, unless the colloids have been formed already.

By forming the colloids by radiation and/or by relative low temperatures, quick curing of the coating can be avoided in an advantageous manner, such that the colloids have sufficient time to form. On the other hand, through the radiation and or heat treatment for the formation of colloids, condensation processes and/or cross-linking reactions occur in the coating that lead to an increase in viscosity, which contributes to the stabilization of the colloids.

The result is a coating with an organically modified inorganic matrix, i.e., in addition to the basic inorganic matrix structure, secondary organic groups are present, which might be and preferably are cross-linked among themselves or via organic compounds. The secondary organic groups are, at least partially, fluorinated and/or copper or silver colloids are present in the matrix, whereby a combination of both features leads to a particularly effective biofilm-inhibiting coating, which exhibits a strong biocide effect, even over longer periods, in particular in connection with liquid media, and excellent anti-adhesion properties.

The following examples illustrate but are not intended to limit the invention.

### EXAMPLES

### 1. Production of a Base Hydrolysate

Introduced into a 1-Liter three-necked flask with thermometer, stirrer and reflux condenser have been 248.4 g (1 mol) of 3-methacryloxypropyltrimethoxysilane (MPTS); while stirring, 99.36 g of acetic acid-stabilized AlO(OH) (Boehmit, Sol P3, Condea company) has been added and suspended for 10 minutes (ratio of MPTS-oxide/AlO(OH)=1.8). Thereafter, the mixture was heated to 90° C. and stirred for 15 more minutes. Then, 35.95 g (2 mol) of distilled water has been added slowly while stirring and the mixture heated to 100° C. After about 5-10 minutes, the reaction mixture foamed vigorously (methanol), whereupon the reaction mixture was heated while stirring for 2.5 hours, calculated from the time of adding the water, at 100° C. oil bath temperature and under reflux. The mixture was then cooled to room temperature and pressure-filtered through a 1 μm membrane filter (cellulose acetate) with a fiberglass pre-filter and then stored at −18° C. until further use.

### 2. Production of an AgNO₃ Solution

27.0 g ethanol has been introduced into a 100 mL round-bottomed flask and 1.28 g (7.5 mol) of AgNO₃ added. The mixture has been stirred for 30 minutes at room temperature and 14.0 g of 2-propanol as well as 3.0 g acetone added. The AgNO₃ solution has been stirred under exclusion of UV-light at room temperature until its use.

### 3. Lacquer 1

143 g of 1-butanol as well as 12.9 g of 1,6-hexanediol dimethacrylate (HDDMA) have been added to 100 g of the filtered base hydrolysate and vigorously stirred for 15 minutes. Thereafter, 1.3 g leveling agent BYKO 306, photo initiator Irgacure® 819 (8.3 g; 5.5 mole percent/mole double bond) as well as 2.6 g fluorine silane (Dynasil® F8261) have been added and stirred for 24 hours under exclusion of UV radiation. The individual components are listed in Table 1 and the weight proportions of the components in Table 2.

### 4. Lacquer 2

100 g of the base hydrolysate have been homogenized with 122.3 g of 1-butanol in a 500-mL three-necked flask with reflux condenser. 23.5 g of the produced AgNO₃ solution has then been added to the mixture and together heated to an oil bath temperature of 60° C. After 10 minutes, 0.72 g of N-(2-aminoethyl)-3-aminopropyltrimethoxysilane has been added in drops while stirring vigorously, whereby the mixture slowly turned brown. The reaction solution has been stirred at 60° C. for 24 hours. After cooling to room temperature, 12.9 g 1,6-hexandiol-dimethacrylate, 1.3 g leveling agent Byk 306 and 8.3 g (5.5 mole percent/mole double bond) photo initiator Irgacure 819 and 2.5 g fluorine silane (Dynasil F8261) have been added and the mixture stirred for 24 hours under exclusion of UV light. The individual components are listed in Table 1 and the weight proportions of the components in Table 2.

TABLE 1

Coating System

| | Lacquer 1 | Lacquer 2 |
|---|---|---|
| Solids content | 35 | 35 |
| [percent in weight] | | |
| Base hydrolysate | 100 g | 100 g |
| 1-butanol | 143.4 g | 122.3 g |
| Comonomer (HDDMA) | 12.9 g | 12.9 g |
| Fluorine silane | 2.6 g | 2.5 g |
| AgNO₃ solution | — | 23.5 g |
| Byk 306 | 1.3 g | 1.3 g |
| Photo initiator | 8.3 g | 8.3 g |

TABLE 2
Weight proportions of the components in the ready to
use lacquer

Coating System
Proportion of the Components in percent
by weight

| | Lacquer 1 | Lacquer 2 |
|---|---|---|
| MPTS oxide | 17.4 | 17.2 |
| AlO(OH) | 9.2 | 9.1 |
| Acetic acid | 0.5 | 0.5 |
| Water | 3.4 | 3.4 |
| Methanol | 9.3 | 9.2 |
| 1-butanol | 53.4 | 45.0 |
| Ethanol | — | 5.2 |
| 2-propanol | — | 2.7 |
| Acetone | — | 0.6 |
| Ag | — | 0.2 |
| DIAMO | — | 0.3 |
| Fluorine silane | 1.0 | 0.9 |
| Irgacure 819 | 3.1 | 3.1 |

### 5. Coatings on PMMA Blend Materials

The described lacquers 1 and 2 have been applied to a PMMA blend material using a brush. After analyzing the leveling properties and after some mechanical tests (e.g., water resistance and weld resistance tests, abrasion resistance) the lacquers have been applied to in-the-ear hearing aid shells. For additional tests (e.g., brushing test or crockmeter test), the coatings have been applied to PMMA blend boards.

### 6. Coatings on Polycarbonate Boards

For a homogenous coating surface (e.g., for angle of contact measurements), the described lacquers 1 and 2 have been applied to planar polycarbonate boards (5×5 cm; 3 mm thickness) using a centrifuge method. A speed of 800 rpm and a centrifuge time of 10 sec resulted in coating thicknesses between 4 and 6 μm (after polymerization).

7. Curing of the Lacquers

The substrates have been provided with a primer of a common lacquer. Curing of the primer and of lacquers 1 and 2 was carried out using blue light (curing system of Dreve, Model Polylux 2×11 W). After a flash-off time of 10 minutes (flash-off temperature: 22° C.), the primer has been cured for 6 minutes under normal atmosphere conditions. After a flash-off time of 10 minutes (flash-off temperature: 22° C.), lacquers 1 and 2 were cured for 2 minutes under an argon atmosphere.

Modifications and substitutions by one of ordinary skill in the art are considered to be within the scope of the present invention which is not to be limited except by the claims which follow.

1. A hearing aid or a hearing aid component for placement in an auditory canal and/or in or behind an auricle of a wearer, having a biofilm-inhibiting coating of an inorganic condensate modified with organic groups on the basis of a coating composition, the biofilm-inhibiting coating including a hydrolysate or pre-condensate of at least one hydrolysable compound with at least one non-hydrolysable substituent, wherein the organic groups of the condensate include at least one part of fluorine atoms and/or copper or silver colloids in the biofilm-inhibiting coating.

2. The hearing aid or a hearing aid component as set forth in claim 1, wherein the hearing aid or a hearing aid component has coated parts, and wherein at least the coated parts are made of a synthetic.

3. The hearing aid or a hearing aid component as set forth in claim 2, wherein the synthetic is a polymethylmethacrylate.

4. The hearing aid or a hearing aid component as set forth in claim 1, further including otoplastic, wherein the otoplastic is a base coating under the biofilm-inhibiting coating.

5. The hearing aid or a hearing aid component as set forth in claim 1, wherein the hydrolysable compounds include at least one hydrolysable silanes with at least one non-hydrolysable substituent.

6. The hearing aid or a hearing aid component as set forth in claim 1, wherein the hydrolysable compounds include at least one silanes according to the general formula (I)
RₐSiX₍₄₋ₐ₎ (I),
wherein the residues R are analogous or different and represent non-hydrolysable groups, the residues X are analogous or different and represent hydrolysable groups or hydroxyl groups and a has a value of 1, 2 or 3.

7. The hearing aid or a hearing aid component as set forth in claim 1, wherein the hydrolysable compounds include at least one silanes that exhibit at least one non-hydrolysable residue, including a functional group.

8. The hearing aid or a hearing aid component as set forth in claim 7, wherein the at least one non-hydrolysable residue is a carbon-carbon double bond.

9. The hearing aid or a hearing aid component as set forth in claim 1, wherein the hydrolysable compounds include at least one silanes of the general formula (II)
Rf®_{b}SiX_{(3-b)} (II),
wherein the residues R are analogous or different and represent non-hydrolysable groups, the residues X are analogous or different and represent hydrolysable groups or hydroxyl groups, Rf is a non-hydrolysable group that exhibits 1 to 30 fluorine atoms bound to carbon atoms, and b is 0, 1 or 2.

10. The hearing aid or a hearing aid component as set forth in claim 1, wherein the coating composition includes copper or silver compounds.

11. The hearing aid or a hearing aid component as set forth in claim 10, wherein the coating composition is copper or silver complex compounds

12. The hearing aid or a hearing aid component as set forth in claim 1, wherein the coating composition has nanoscale inorganic particles therein.

13. The hearing aid or a hearing aid component as set forth in claim 1, wherein the biofilm-inhibiting coating is made by applying the coating composition to at least a portion of a surface of the hearing aid or component of the hearing aid and by heating.

14. The hearing aid or a hearing aid component as set forth in claim 1, wherein the biofilm-inhibiting coating is made by applying the coating composition to at least a portion of a surface of the hearing aid or component of the hearing aid and by treating it with radiation.

15. The hearing aid or a hearing aid component as set forth in claim 10, wherein the copper or silver compounds in the coating composition are converted to copper or silver colloids through heat.

16. The hearing aid or a hearing aid component as set forth in claim 11, wherein the copper or silver compounds in the coating composition are converted to copper or silver colloids through radiation treatment.

17. The hearing aid or a hearing aid component as set forth in claim 1, wherein the biofilm-inhibiting coating is obtained by applying the coating composition having a copper or silver compound therein, and by heating under formation of the copper or silver colloid-containing coating.

18. The hearing aid or a hearing aid component as set forth in claim 1, wherein the biofilm-inhibiting coating is obtained by applying the coating composition having a copper or silver compound therein, and by radiating under formation of the copper or silver colloid-containing coating.

19. The hearing aid or a hearing aid component as set forth in claim 1, wherein a portion of the organic groups of the condensate includes fluorine atoms and copper or silver colloids within the coating.
